(19) United States
(12) Patent Application Publication
(10) Pub. No.: US 2022/0226323 A1
(43) Pub. Date: Jul. 21, 2022

(54) ALDOSE REDUCTASE INHIBITORS FOR TREATMENT OF PHOSPHOMANNOMUTASE 2 DEFICIENCY

(71) Applicant: Applied Therapeutics, Inc., New York, NY (US)

(72) Inventors: Riccardo Perfetti, New York, NY (US); Shoshana SHENDELMAN, New York, NY (US)

(21) Appl. No.: 17/715,401

(22) Filed: Apr. 7, 2022

Related U.S. Application Data
(63) Continuation of application No. PCT/US2020/054607, filed on Oct. 7, 2020.
(60) Provisional application No. 62/912,441, filed on Oct. 8, 2019.

(51) Int. Cl. A61K 31/5025

(52) U.S. Cl. CPC A61K 31/5025

(57) ABSTRACT

The disclosure relates to methods for treating PMM2-CDG using aldose reductase inhibitors.

### RELATED APPLICATIONS

This application claims the benefit of U.S. Provisional Application No. 62/912,441 filed on Oct. 8, 2019, the entire disclosure of which is incorporated herein by reference.

### BACKGROUND

Phosphomannomutase 2 (PMM2) is an enzyme that converts mannose-6-phosphate (M6P) into mannose-1-phosphate (M1P). M1P is a precursor to GDP-mannose, which is necessary for the production of dalichol-P-oligosaccharides that are important for protein glycosylation.

PMM2 forms an obligate homodimer in the cytoplasm and converts mannose-6-phosphate to mannose-1-phosphate. Each PMM2 monomer forms a dimer with itself as a prerequisite for catalytic activity, although there need only be one functional active site per dimer (Andreotti, G., et. al., 2015, PLoS ONE 10, e0139882. doi:10.1371/journal.pone.013988). Glucose-1,6-bisphosphate and mannose-1,6-bisphosphate are endogenous coactivators of PMM2 function, binding to and stabilizing PMM2 dimers (Id.).

PMM2 deficiency is responsible for the most common congenital disorder of glycosylation (CDG) (Van, S. E. et al., FEBS Lett. 1995, 377, 318-320, Ferreira, C. R. et al., J. Inherit. Metab. Dis. 2018, 41, 541-553). PMM2-CDG is a multisystem, multi-organ disease because a minimal level of glycosylation is required at all times in all cells of the body, with different cell types and organs more or less vulnerable to the complex sequelae of hypoglycosylation. As the residual level of PMM2 enzymatic activity increases, the number and severity of organ systems affected decreases. Mutations in the gene encoding PMM2 are responsible for PMM2-CDG (Jaeken, J. et al., J Inherit Metab Dis. 2008, 31, 669-72), and more than 115 mutations in the PMM2 gene have been found that cause PMM2-CDG. All disease causing mutations appear to reduce the enzymatic activity of PMM2, leading to an insufficient amount of activated mannose to form oligosaccharides for normal protein glycosylation. PMM2-CDG is also known as CDG-1A or Jaeken syndrome. PMM2-CDG displays variable clinical progression and presentation, with affected individuals typically developing signs and symptoms during infancy. Organs that are affected by PMM2-CDG include brain, liver, gastrointestinal tract, heart and kidney. About 20% of affected infants die before 1 year of age due to multiple organ failure. The most severe cases of PMM2-CDG are characterized by hydrops fetalis, and in most cases babies with hydrops fetalis are stillborn or die soon after birth. Most PMM2-CDG patients who survive infancy have intellectual disability and developmental delay (Schiff, M. et al., J Med Genet., 2017, 54, 843-851).

Currently there are no therapeutic approaches for treating PMM2-CDG that are effective, and the disease in managed by efforts to reduce disease manifestations (e.g., occupational, physical and speech therapy). Accordingly, there is a recognized but unmet need for methods for treating PMM2-CDG.

### SUMMARY

This disclosure relates to methods for treating PMM2-CDG by administering a therapeutically effective amount of an Aldose Reducatase (AR) inhibitor to a subject in need thereof. Without wishing to be bound by any particular theory, it is believed that inhibition of AR can potentiate PMM2 enzymatic activity.

In one example, the method for the treatment of PMM2-CDG comprises administering to a subject in need thereof a therapeutically effective amount of zopolrestat. In one example, the method for the treatment of PMM2-CDG comprises administering to a subject in need thereof a therapeutically effective amount of a compound of any one of Formulas (I)-(VI). In some aspects, the AR inhibitor administered is not ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210. In one example, the method for the treatment or prevention of PMM2-CDG excludes the administration of Epalrestat. In other examples, the method for the treatment or prevention of PMM2-CDG excludes the administration of Epalrestat and alpha-cyano-4-hydroxycinnamic acid.

The subject to be treated in accordance with the methods disclosed herein can have classical pediatric clinical presentations such as developmental delay, severe encephalopathy with axial hypotonia, abnormal eye movements, psychomotor retardation and/or cerebellar hypoplasia. The subject to be treated in accordance with the methods disclosed herein can have hypogonadism, coagulation abnormalities and thrombotic events, retinitis pigmentosa and/or peripheral neuropathy.

In other embodiments, the disclosure relates to a method of treating PMM2-CDG in a subject in need thereof comprising, administering a therapeutically effective amount of a pharmaceutical composition comprising AR inhibitor, such as a compound of any one of Formulas (I)-(VI), and a pharmaceutically acceptable carrier. The disclosure relates to a method of increasing PMM2 enzymatic activity in a subject with PMM2-CDG, comprising administering a therapeutically effective amount of an aldose reductase inhibitor, such as a compound of any one of Formulas (I)-(VI), to the subject.

In other embodiments, the disclosure relates to a method of treating PMM2-CDG in a subject in need thereof comprising, administering an therapeutically effective amount of

(a) a compound of Formulas (I)-(VI) and a pharmaceutically acceptable carrier; and

(b) one or more of alponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210.

In other embodiments, this disclosure relates to the use of an AR inhibitor for increasing PMM2 enzymatic activity for therapy of PMM2-CDG.

In other embodiments, this disclosure relates to the use of an AR inhibitor for the manufacture of a medicament for treating PMM2-CDG.

The disclosure also relates to the use of an AR inhibitor (e.g., zopolrestat, epalrestat, compound of any one of Formulas (I)-(VI)) for the treatment of PMM2-CDG.

The disclosure also relates to an AR inhibitor (e.g., zopolrestat, epalrestat, compound of any one of Formulas (I)-(VI)) for the manufacture of a medicament for the treatment of PMM2-CDG.

The disclosure also relates to a pharmaceutical formulation for the treatment of PMM2-CDG, that contains an AR inhibitor (e.g., zopolrestat, epalrestat, compound of any one of Formulas (I)-(VI) as an active ingredient.

### BRIEF DESCRIPTION OF DRAWINGS

FIG. 1 illustrates the in vitro inhibition of Aldose Reductase by Compound B.

FIG. 2 illustrates the activation of PMM2 activity in PMM2-CDG patient-derived fibroblasts treated with Compound B.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete.

This disclosure relates to the use of AR inhibitors for the treatment of PMM2-CDG.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed, as well as the range of values greater than or equal to 1 μM and the range of values less than or equal to 8 μM.

The singular forms “a,” “an,” and “the” include plural referents unless the context clearly dictates otherwise. Thus, for example, reference to a “compound of Formula (I)” includes a single compound as well as two or more of the same or different compounds; reference to an “excipient” includes a single excipient as well as two or more of the same or different excipients, and the like.

The word “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

In order to provide a complete, concise and clear description of the various embodiments, this disclosure includes descriptions of various components, groups of components, ranges and other elements of the broader disclosure. It is intended that such elements can be variously combined to provide additional embodiments of the disclosure. It is also intended that any disclosed features (e.g., substituent, analog, compound, structure, component) including individual members of any disclosed group, including any sub-ranges or combinations of sub-ranges within the group, may be excluded from the disclosure or any embodiments of the disclosure for any reason.

The various embodiments of the present disclosure are further described in detail in the numbered paragraphs below.

### I. Methods

In general, the disclosure relates to a method for the treatment of PMM2-CDG, comprising administering to a subject in need thereof a therapeutically effective amount of a compound that inhibits aldose reductase activity. The compound can be any suitable compound that inhibits AR activity, such as a small molecule compound (e.g., having a size of 5 kDa or less), a biologic agent (e.g., an inhibitory RNA directed against aldose reductase) or a combination thereof. Preferably, the AR inhibitor is a small molecule compound. Suitable small molecule AR inhibitors are known in the art and are disclosed herein. Small molecule AR inhibitors include ponalrestat, sorbinil, sorbinol, imirestat, AND-138, CT-112, zenarestat, BAL-AR18, AD-5467, M-79175, tolrestat, alconil, statil, berberine, SPR-210 zopolrestat, epalrestat, the compounds disclosed in U.S. Pat. Nos. 8,916,563, 9,650,383, 10,150,779 and the compounds disclosed herein. alpha-cyano-4-hydroxycinnamic acid is also an AR inhibitor. Preferred AR inhibitors for use in the invention include zopolrestat, epalrestat, the compounds disclosed in U.S. Pat. Nos. 8,916,563, 9,650,383, 10,150,779 and the compounds disclosed herein. The AR inhibitors can be administered in any suitable molecular form including pharmaceutically acceptable salts, solvates, prodrugs, and compounds that contain stable isotopic forms of one or more atoms, e.g., deuterium in place of hydrogen.

In one example, the method for the treatment of PMM2-CDG comprises administering to a subject in need thereof a therapeutically effective amount of zopolrestat.

In one example, the method for the treatment of PMM2-CDG comprises administering to a subject in need thereof an therapeutically effective amount of epalrestat.

In one example, the method for the treatment of PMM2-CDG comprises administering to a subject in need thereof an therapeutically effective amount of an aldose reductase, wherein the aldose reductase inhibitor is not ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210. In particular embodiments, the methods for the treatment of PMM2-CDG disclosed herein do not include administering epalrestat. In particular embodiments, the methods for the treatment of PMM2-CDG disclosed herein do not include administering epalrestat or alpha-cyano-4-hydroxycinnamic acid.

In one example, the method for the treatment of PMM2-CDG comprises administering to a subject in need thereof an therapeutically effective amount of a compound of any one of Formulas (I)-(VI). In certain examples, the compound that is administered is Compound A or the compound that is administered is Compound B or a physiologically acceptable salt, hydrate, solvate or prodrug of Compound A or Compund B.

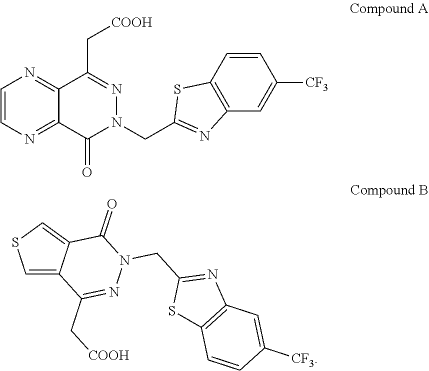

As used herein, the term “treating” refers to curative or palliative (e.g., control or mitigate a disease or disease symptoms) therapy. This can include reversing, reducing, arresting or delaying the symptoms, clinical signs, and underlying pathology of PMM2-CDG in a manner to improve or stabilize a subject's condition. Thus, the method can be used for treatment of PMM2-CDG, treatment of complications (e.g., symptoms and clinical signs) of PMM2-CDG, and/or treatment and prevention of complications (e.g., symptoms and clinical signs) of PMM2-CDG.

As used herein “a therapeutically effective amount” is an amount of a compound that is sufficient to achieve the desired therapeutic effect under the conditions of administration, such as an amount that reduces or ameliorates the severity of PMM2-CDG, that prevents the advancement of conditions or symptoms related to PMM2-CDG, or enhances or otherwise improves therapeutic effect(s) of another therapy for the treatment or management of PMM2-CDG. A therapeutically effective amount can be an amount that increases PMM2 enzymatic activity, in the subject being treated. The actual amount administered can be determined by an ordinarily skilled clinician based upon, for example, the subjects age, weight, sex, general heath and tolerance to drugs, severity of disease, dosage form selected, route of administration and other factors. Typically, the amount of an AR inhibitor that is administered is from about 0.5 to about 60 mg/kg body weight per day, such as from about 1.0 to 10 mg/kg.

In some examples of the practice of the methods disclosed herein, the therapeutically effective amount is an amount sufficient to reduce intracellular aldose reductase activity at least by about 20%, about 30%, about 40%, about 50%, about 60%, about 70%, about 80%, about 90%, about 95%, about 99%, or more, e.g., about 100% (e.g., compared to pre-treatment level). The therapeutically effective amount can be an amount that increases PMM2 enzymatic activity at least by about 20%, about 30%, about 40%, about 50%, about 60%, about 70%, about 80%, about 90%, about 95%, about 99%, or more, e.g., about 100% (e.g., compared to pre-treatment level). The therapeutically effective amount can be sufficient to restore PMM2 enzyme levels in a subject with PMM2-CDG.

A “subject” can be any animal with PMM2-CDG, particularly a mammal, and including, but by no means limited to, humans, domestic animals, such as feline or canine subjects, farm animals, such as but not limited to bovine, equine, caprine, ovine, avian and porcine subjects, wild animals (whether in the wild or in a zoological garden), research or laboratory animals, such as mice, rats, rabbits, goats, sheep, pigs, dogs, cats, etc., avian species, such as chickens, turkeys, songbirds, and the like. Typically, a human subject to be treated using the methods disclosed herein is diagnosed with PMM2-CDG as a new born through enzymatic or genetic screening, and has deficiency in PMM2 activity.

This disclosure also relates to the prophylaxis or treatment of at least one clinical feature or complication of PMM2-CDG in a subject. Representative clinical features or complications which can be present in children, adolescents or adults, include, e.g., alternating internal strabism and other abnormal eye movements, axial hypotonia, intellectual disability, ataxia, and hyporeflexia. After infancy, symptoms include retinitis pigmentosa, often stroke-like episodes, and sometimes epilepsy. Other features are variable dysmorphy (large, hypoplastic/dysplastic ears), abnormal subcutaneous adipose-tissue distribution (fat pads, inverted nipples), mild to moderate hepatomegaly, skeletal abnormalities (including atlantoaxial subluxation), and hypogonadism. Some infants develop pericardial effusion and/or cardiomyopathy. At the other end of the clinical spectrum are patients with a very mild phenotype (no dysmorphy, very mild intellectual disability, ataxia) (Jaeken, J. et al, “Glycosylation and its Disorders: General Overview,” Elsevier, Reference Module in Biomedical Sciences, 2016).

In a particular aspect, the disclosure relates to a method for the treatment of a clinical feature or complication of PMM2-CDG and comprises administering to a subject in need thereof a therapeutically effective amount of zopolrestat.

In one example, the disclosure relates to a method for the treatment of a clinical feature or complication of PMM2-CDG and comprises administering to a subject in need thereof a therapeutically effective amount of epalrestat.

In one example, the disclosure relates to a method for the treatment of a clinical feature or complication of PMM2-CDG and comprises administering to a subject in need thereof a therapeutically effective amount of a compound of any one of Formulas (I)-(VI).

In some embodiments, the aforementioned methods are carried out by administering a formulation comprising of one or more AR inhibitors. The formulations can be adapted for administration once daily, twice daily, three times daily or four times daily to a subject in need thereof for the desired treatment period. Typically, the formulations are adapted for chronic administration over the course of several weeks, months, years or decades. In still other embodiments, the methods are carried out by administering formulations that are adapted for administration over the course of several weeks. Typically, the methods are carried out by administering formulations that are adapted for administration over the course of several years or decades.

### II. AR Inhibitors

Suitable small molecule AR inhibitors are known in the art and are disclosed herein. Small molecule AR inhibitors include ponalrestat, sorbinil, sorbinol, imirestat, AND-138, CT-112, zenarestat, BAL-AR18, AD-5467, M-79175, tolrestat, alconil, statil, berberine, SPR-210, zopolrestat, epalrestat, the compounds disclosed in U.S. Pat. Nos. 8,916,563, 9,650,383, WO2012/009553 and the compounds disclosed herein. Preferred AR inhibitors for use in the invention zopolrestat, epalrestat, the compounds disclosed in U.S. Pat. Nos. 8,916,563, 9,650,383, WO 2017/038505, U.S. Pat. No. 10,150,779 and the compounds disclosed herein. The disclosures of U.S. Pat. Nos. 8,916,563, 9,650,383, 10,150,779, WO 2012/009553, and WO 2017/038505 are incorporated by reference herein in their entirety, and disclose compounds that are suitable for use in the methods described herein.

### Compounds of Formulas (I) and (II)

In one example, the AR inhibitor is a compound of Formula (I) or pharmaceutically acceptable salts, prodrugs and solvates thereof,

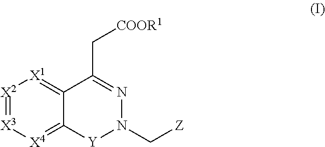

wherein,

R¹ is H, (C₁-C₆)-alkyl, (C₁-C₆)-hydroxyalkyl, or (C₁-C₆)-aminoalkyl;

X¹ is N or CR³;

X² is N or CR⁴;

X³ is N or CR⁵;

X⁴ is N or CR⁶; with the proviso that two or three of X¹, X², X³, or X⁴ are N;

Y is a bond, C═O, C═S, C═NH, or C═N(C₁-C₄)-alkyl;

Z is

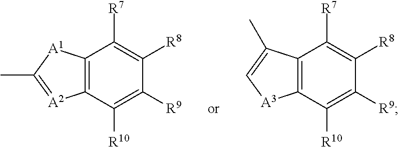

A¹ is NR¹¹, O, S or CH₂;

A² is N or CH;

A³ is NR¹¹, O, or S;

R³ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R³ through R⁶ or two of R⁷ through R¹⁰ taken together are (C₁-C₄)-alkylenedioxy; and

R¹¹ is hydrogen, C₁-C₄ alkyl, or C(O)O-(C₁-C₄)-alkyl.

It will be recognized by those of skill in the art that the designation of Z is

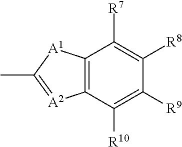

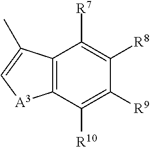

or Z is

indicates that when Z is

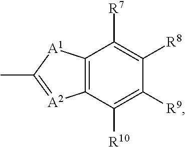

the compounds of formula (I) encompass

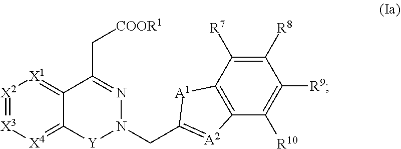

and when Z is

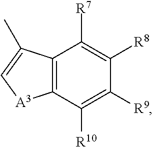

the compounds of formula (I) encompass

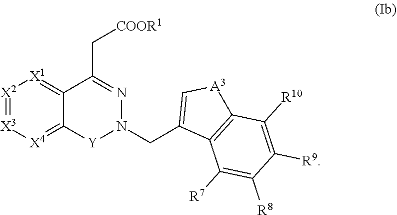

In certain embodiments, R¹ is hydrogen or (C₁-C₆)-alkyl. In certain embodiments, R¹ is hydrogen. In certain embodiments, R¹ is (C₁-C₆)-alkyl. In certain embodiments, R¹ is tert-butyl.

In certain embodiments, R³ through R¹⁰ are independently hydrogen, halogen or haloalkyl. In certain embodiments, R³ through R¹⁰ are independently hydrogen, halogen or trihaloalkyl.

In certain embodiments, R³ through R⁶ are hydrogen.

In certain embodiments, R⁷ through R¹⁰ are independently hydrogen, halogen or haloalkyl. In certain embodiments, R⁷ through R¹⁰ are independently hydrogen, halogen or trihaloalkyl.

In certain embodiments, R⁷ and R¹⁰ are hydrogen.

In certain embodiments, R⁸ is hydrogen, halogen or haloalkyl. In certain embodiments, R⁸ is hydrogen. In certain embodiments, R⁸ is halogen. In certain embodiments, R⁸ is haloalkyl.

In certain embodiments, R⁹ is hydrogen, halogen or haloalkyl. In certain embodiments, R⁹ is hydrogen. In certain embodiments, R⁹ is halogen. In certain embodiments, R⁹ is haloalkyl.

In certain embodiments, Y is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A¹ is NR¹¹, S or CH₂. In certain embodiments, A¹ is NR¹¹ or O. In certain embodiments, A¹ is NR¹¹ or S. In certain embodiments, A¹ is NR¹¹. In certain embodiments, A¹ is O. In certain embodiments, A¹ is S.

In certain embodiments, A² is N or CH. In certain embodiments, A¹ is N. In certain embodiments, A¹ is CH.

In certain embodiments, A³ is O or S. In certain embodiments, A³ is O.

In certain embodiments, A³ is S.

In certain embodiments, X¹ and X⁴ are nitrogen.

In certain embodiments, X¹ and X² are nitrogen.

In certain embodiments, X¹ and X³ are nitrogen.

In certain embodiments, X² and X³ are nitrogen.

In certain embodiments, X² and X⁴ are nitrogen.

In certain embodiments, X³ and X⁴ are nitrogen.

In certain embodiments, Z is

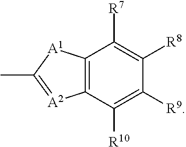

In certain embodiments, Z is

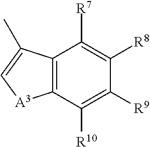

In certain embodiments, R¹ is hydrogen or (C₁-C₆)-alkyl;

X¹ and X⁴ are N;

X² is CR⁴;

X³ is CR⁵;

Y is C═O;

Z is

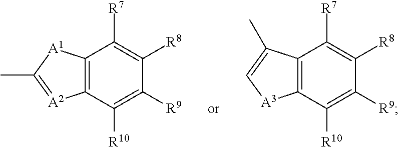

A¹ is NR¹¹, O, or S;

A² is N;

A³ is O, or S;

R⁴ and R⁵ are hydrogen;

R⁷ through R¹⁰ are independently hydrogen, halogen, cyano, acyl, haloalkyl, haloalkoxy, haloalkylthio, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and

R¹¹ is hydrogen, C₁-C₄ alkyl, or C(O)O—(C₁-C₄)-alkyl.

In certain embodiments, R¹ is hydrogen or tert-butyl;

X¹ and X⁴ are N;

X² is CR⁴;

X³ is CR⁵;

Y is C═O;

Z is

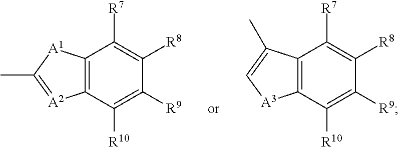

A¹ is NR¹¹, O or S;

A² is N;

A³ is O or S;

R⁴ and R⁵ are hydrogen;

R⁷ through R¹⁰ are independently hydrogen, halogen, or haloalkyl; and

R¹¹ is hydrogen, (C₁-C₄)-alkyl, or C(O)O-tert-butyl.

In certain embodiments, R¹ is hydrogen or tert-butyl;

X¹ and X⁴ are N;

X² is CH;

X³ is CH;

Y is C═O;

Z is

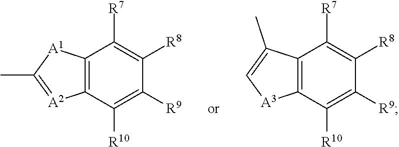

A¹ is NR¹¹, O or S;

A² is N;

A³ is O or S;

R⁷, R⁸ and R¹⁰ are independently hydrogen, halogen, or haloalkyl;

R⁹ is halogen, or haloalkyl; and

R¹¹ is hydrogen or methyl.

In certain embodiments, R¹ is hydrogen or tert-butyl;

X¹ and X⁴ are N;

X² is CH;

X³ is CH;

Y is C═O;

Z is

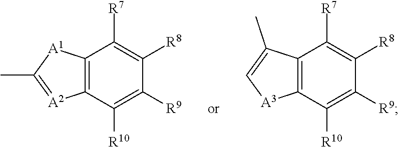

A¹ is NR¹¹, O or S;

A² is N;

A³ is O or S;

R⁷, R⁸ and R¹⁰ are independently hydrogen, halogen, or haloalkyl;

R⁹ is chlorine, or trifluoromethyl; and

R¹¹ is hydrogen or methyl.

In certain embodiments, the AR inhibitor is a compound of Formula (II) or pharmaceutically acceptable salt or solvate thereof:

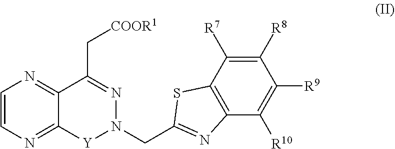

Wherein R¹, R⁷-R⁹ and Y are as described in Formula (I), and preferable wherein R¹ is hydrogen or (C₁-C₆)-alkyl and Y is C═O. Exemplary compounds of Formula (II) include the following and salts thereof:

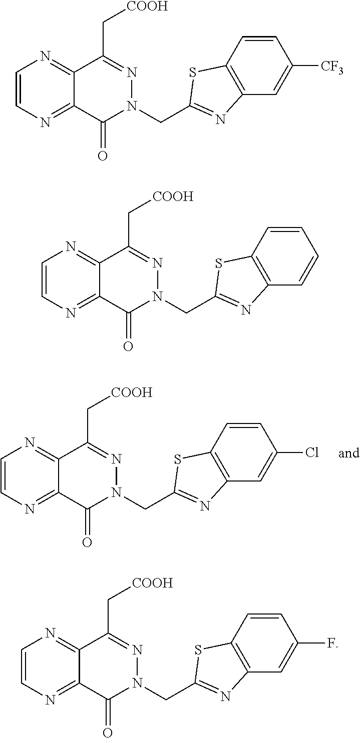 [SMILES: O=C(O)Cc1nn(Cc2nc3cc(C(F)(F)F)ccc3s2)c(=O)c2nccnc12 ; O=C(O)Cc1nn(Cc2nc3ccccc3s2)c(=O)c2nccnc12 ; O=C(O)Cc1nn(Cc2nc3cc(Cl)ccc3s2)c(=O)c2nccnc12 ; O=C(O)Cc1nn(Cc2nc3cc(F)ccc3s2)c(=O)c2nccnc12]

### Compounds of Formula (III)

The AR inhibitors can be a compound of Formula (III) or pharmaceutically acceptable salts, pro-drugs and solvates thereof,

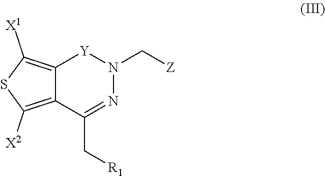

wherein,
R¹ is CO₂R² or CO₂⁻X⁺;
R² is H, (C₁-C₆)-alkyl, (C₁-C₆)-hydroxyalkyl, or (C₁-C₆)-aminoalkyl;
X¹ is H or halogen;
X² is H or halogen;
Y is a bond, C═O, C═S, C═NH, or C═N(C₁-C₄)-alkyl;
Z is

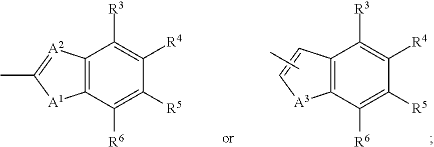

A¹ is NR⁷, O, S or CH₂;
A² is N or CH;
A³ is NR⁷, O, or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl;
R⁷ is hydrogen, C₁-C₄ alkyl, or C(O)O-(C₁-C₄)-alkyl; and
X⁺ is a counter ion.
It will be recognized by those of skill in the art that the designation of

Z is

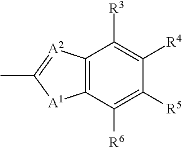

### or Z is

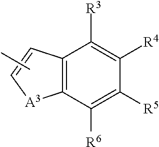

indicates that when Z is

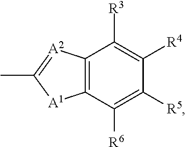

the compounds of Formula (III) are understood to encompass

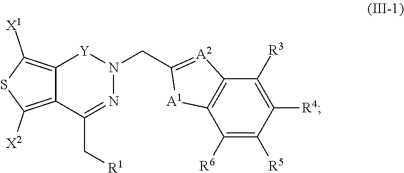

and when Z is

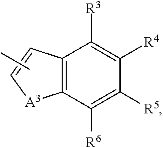

the compounds of Formula (I) are understood to encompass

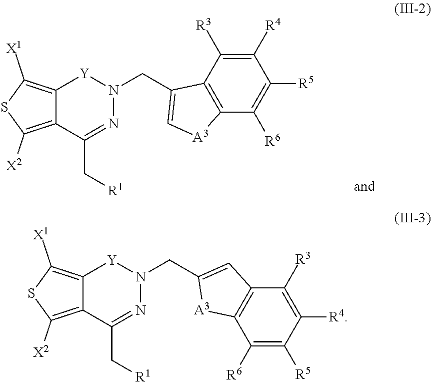

In certain embodiments, R¹ is CO₂R² or CO₂⁻X⁺. In certain embodiments, R¹ is CO₂R². In certain embodiments, R¹ is CO₂⁻X⁺.

In certain embodiments, R² is hydrogen or (C₁-C₆)-alkyl. In certain embodiments, R² is hydrogen or (C₁-C₄)-alkyl. In certain embodiments, R² is hydrogen or (C₁-C₃)-alkyl. In certain embodiments, R² is hydrogen, methyl, or ethyl. In certain embodiments, R² is hydrogen or methyl. In certain embodiments, R² is methyl or ethyl. In certain embodiments, R² is methyl. In certain embodiments, R² is hydrogen. In certain embodiments, R² is (C₁-C₆)-alkyl. In certain embodiments, R² is (C₁-C₆)-n-alkyl. In certain embodiments, R² is (C₁-C₂)-alkyl. In certain embodiments, R² is (C₁-C₃)-alkyl. In certain embodiments, R² is (C₁-C₄)-alkyl. In certain embodiments, R² is tert-butyl.

In certain embodiments, R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl.

In certain embodiments, R³ through R⁶ are independently hydrogen, halogen or haloalkyl. In certain embodiments, R³ through R⁶ are independently hydrogen, halogen or trihaloalkyl.

In certain embodiments, R³ and R⁶ are hydrogen. In certain embodiments, R³, R⁵, and R⁶ are hydrogen.

In certain embodiments, R⁴ is hydrogen, halogen or haloalkyl. In certain embodiments, R⁴ is hydrogen. In certain embodiments, R⁴ is halogen. In certain embodiments, R⁴ is haloalkyl. I n certain embodiments, R⁴ is CF₃.

In certain embodiments, R³ through R⁶ are hydrogen. In certain embodiments, R³, R⁵, R⁶ are hydrogen and R⁴ is halogen or haloalkyl. In certain embodiments, R³, R⁵, R⁶ are hydrogen and R⁴ is haloalkyl. In certain embodiments, R³, R⁵, R⁶ are hydrogen and R⁴ is CF₃. In certain embodiments, R³, R⁵, R⁶ are hydrogen and R⁴ is halogen. In certain embodiments, R³, R⁵, R⁶ are hydrogen and R⁴ is F. In certain embodiments, R³, R⁵, R⁶ are hydrogen and R⁴ is Cl.

In certain embodiments, Y is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄-alkyl.

In certain embodiments, A¹ is NR⁷, O, S or CH₂. In certain embodiments, A¹ is NR⁷, O, or S. In certain embodiments, A¹ is NR⁷, S or CH₂. In certain embodiments, A¹ is NR⁷ or O. In certain embodiments, A¹ is NR⁷ or S. In certain embodiments, A¹ is NR⁷. In certain embodiments, A¹ is O. In certain embodiments, A¹ is S.

In certain embodiments, A² is N or CH. In certain embodiments, A² is N. In certain embodiments, A² is CH.

In certain embodiments, A³ is NR⁷, O, or S. In certain embodiments, A³ is O. In certain embodiments, A³ is S. In certain embodiments, A³ is NR⁷.

In certain embodiments, X¹ and X² are hydrogen.

In certain embodiments, X¹ and X² are halogen. In certain embodiments, X¹ and X² are Cl.

In certain embodiments, X¹ and X² are independently hydrogen or halogen. In certain embodiments, X¹ is hydrogen and X² is Cl. In certain embodiments, X¹ is Cl and X² is hydrogen.

In certain embodiments, Z is

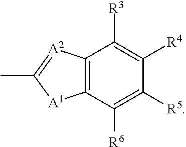

In certain embodiments, Z is

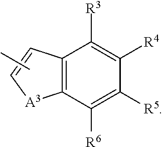

In certain embodiments, R⁷ is hydrogen, C₁-C₄ alkyl, or C(O)O-(C₁-C₄)-alkyl. In certain embodiments, R⁷ is hydrogen. In certain embodiments, R⁷ is C₁-C₄ alkyl. In certain embodiments, R⁷ is C₁-C3 alkyl. In certain embodiments, R⁷ is C₁-C2 alkyl. In certain embodiments, R⁷ is C₁-C₄ n-alkyl. In certain embodiments, R⁷ is C₁-C₃ n-alkyl. In certain embodiments, R⁷ is C(O)O-(C₁-C₄)-alkyl. In certain embodiments, R⁷ is C(O)O-(C₁-C₃)-alkyl. In certain embodiments, R⁷ is C(O)O-(C₁-C₂)-alkyl. In certain embodiments, R⁷ is C(O)O-(C₁-C₄)-n-alkyl. In certain embodiments, R⁷ is C(O)O-(C₁-C₃)-n-alkyl.

In certain embodiments, R¹ is CO₂R²;
R² is H or (C₁-C₆)-alkyl;
X¹ is H;
X² is H;
Y is C═O;
Z is

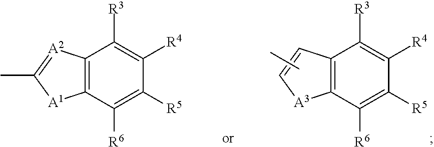

A¹ is NR⁷, O, or S;
A² is N;
A³ is O or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and
R⁷ is hydrogen, C₁-C₄ alkyl, or C(O)O-(C₁-C₄)-alkyl.

In certain embodiments, R¹ is CO₂R²;
R² is H or tert-butyl;
X¹ is H;
X² is H;
Y is C═O;
Z is

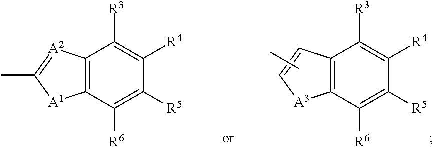

A¹ is NR⁷, O, or S;
A² is N;
A³ is O or S;
R⁶ through R⁶ are independently hydrogen, halogen, haloalkyl; and
R⁷ is hydrogen, C₁-C₄ alkyl, or C(O)O-(C₁-C₄)-alkyl.
In certain embodiments, R¹ is CO₂R²;
R² is H or tert-butyl;
X¹ is H;
X² is H;
Y is C═O;
Z is

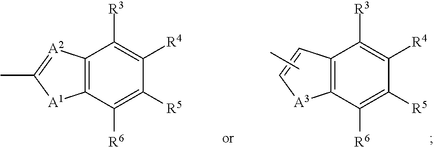

A¹ is NR⁷, O, or S;
A² is N;
A³ is O or S;
R³, R⁵, and R⁶ are hydrogen;
R⁴ is hydrogen, halogen, or haloalkyl; and
R⁷ is hydrogen, C₁-C₄ alkyl, or C(O)O-(C₁-C₄)-alkyl.
In certain embodiments, R¹ is CO₂R²;
R² is H or (C₁-C₆)-alkyl;
X¹ is halogen;
X² is halogen;
Y is C═O;
Z is

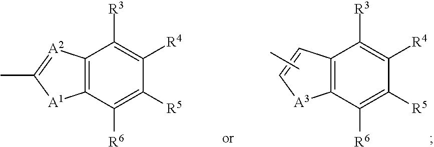

A¹ is NR⁷, O, or S;
A² is N;
A³ is O or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and
R⁷ is hydrogen, C₁-C₄ alkyl, or C(O)O-(C₁-C₄)-alkyl.
In certain embodiments, R¹ is CO₂R²;
R² is H or tert-butyl;
X¹ is halogen;
X² is halogen;
Y is C═O;
Z is

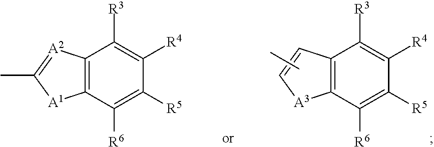

A¹ is NR⁷, O, or S;
A² is N;
A³ is O or S;
R³ through R⁶ are independently hydrogen, halogen, haloalkyl; and
R⁷ is hydrogen, C₁-C₄ alkyl, or C(O)O-(C₁-C₄)-alkyl.
In certain embodiments, R¹ is CO₂R²;
R² is H or tert-butyl;
X¹ is Cl;
X² is Cl;
Y is C═O;
Z is

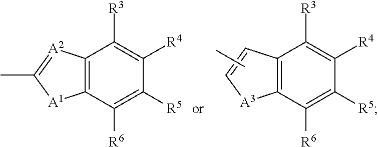

A¹ is NR⁷, O, or S;
A² is N;
A³ is O or S;
R³ through R⁶ are independently hydrogen, halogen, haloalkyl; and
R⁷ is hydrogen, C₁-C₄ alkyl, or C(O)O-(C₁-C₄)-alkyl.
In certain embodiments, R¹ is CO₂R²;
R² is H or tert-butyl;
X¹ is Cl;
X² is Cl;
Y is C═O;
Z is

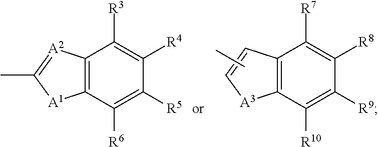

A¹ is NR⁷, O, or S;
A² is N;
A³ is O or S;
R³, R⁵, and R⁶ are hydrogen;
R⁴ is hydrogen, halogen, or haloalkyl; and
R⁷ is hydrogen, C₁-C₄ alkyl, or C(O)O-(C₁-C₄)-alkyl.
In certain embodiments, the compound of Formula (III) is selected from the group consisting of:

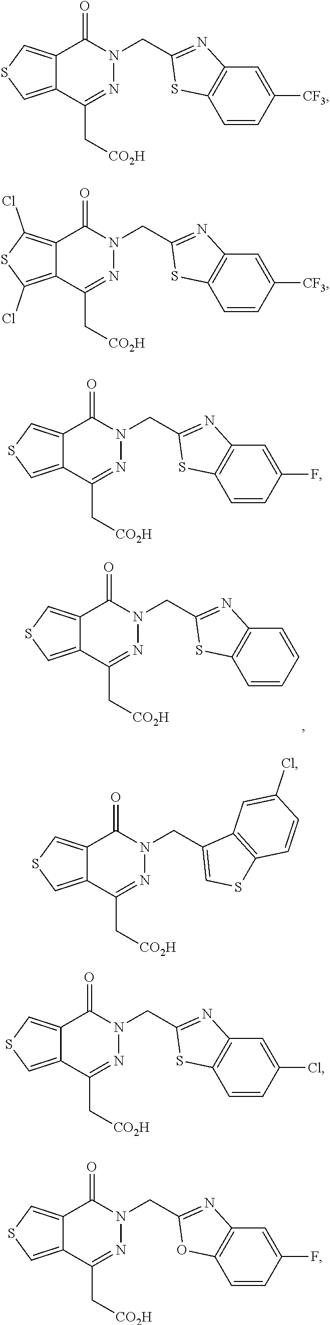

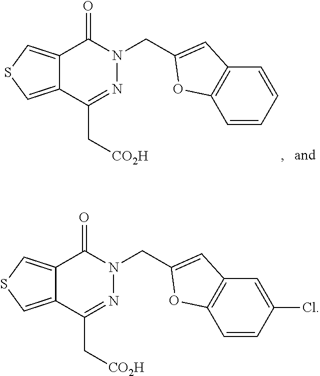 [SMILES: O=C(O)Cc1nn(Cc2cc3ccccc3o2)c(=O)c2cscc12 ; O=C(O)Cc1nn(Cc2cc3cc(Cl)ccc3o2)c(=O)c2cscc12]

In certain embodiments, the compound of Formula (III) is

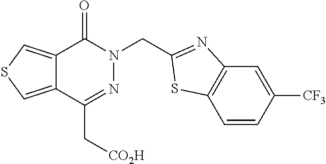 [SMILES: O=C(O)Cc1nn(Cc2nc3cc(C(F)(F)F)ccc3s2)c(=O)c2cscc12]

or a pharmaceutically acceptable salt thereof.
In certain embodiments, the compound of Formula (III) is

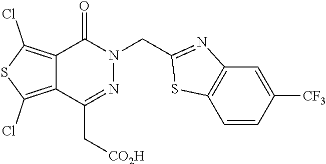 [SMILES: O=C(O)Cc1nn(Cc2nc3cc(C(F)(F)F)ccc3s2)c(=O)c2c(Cl)sc(Cl)c12]

or a pharmaceutically acceptable salt thereof.

### Compounds of Formulas (IV), (V) and (VI)

The AR inhibitors can be a compound of Formula (IV) or pharmaceutically acceptable salts, and solvates thereof,

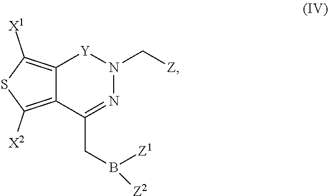

wherein,

X¹ is H or halogen;

X² is H or halogen;

Y is a bond, C═O, C═S, C═NH, or C═N(C₁-C₄)-alkyl;

Z¹ and Z² are independently selected from the group consisting of hydroxy, alkoxy, aryloxy, or Z¹ and Z² taken together with the boron atom to which they are bonded form

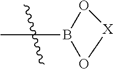

wherein,

X is a substituted or unsubstituted C₂-C₅ alkylene;

Z is

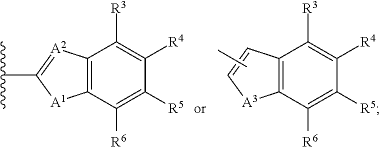

A¹ is NR⁷, O, S or CH₂;

A² is N or CH;

A³ is NR⁷, O, or S;

R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and

R⁷ is hydrogen, C₁-C₄ alkyl, or C(O)O-(C₁-C₄)-alkyl.

Suitable substituents on the C₂-C₅ alkylene include one or more alkyl, alkoxy, aryl, aryloxy, halo, haloalkyl, haloalkoxy, haloalkylthio. A preferred substituted C₂-C₅ alkylene is substituted ethylene. A more preferred substituted C₂-C₅ alkylene is —C(CH₃)₂C(CH₃)₂—.

It will be recognized by those of skill in the art that the designation of
Z is

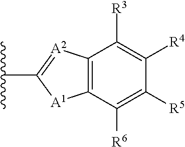

### or Z is

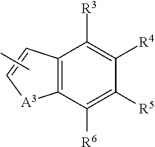

indicates that when Z is

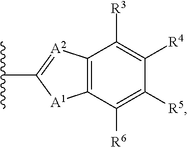

the compounds of Formula (IV) are understood to encompass

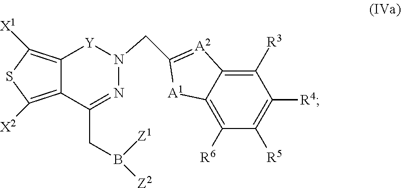

and

when Z is

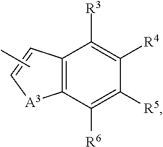

the compounds of Formula (IV) are understood to encompass

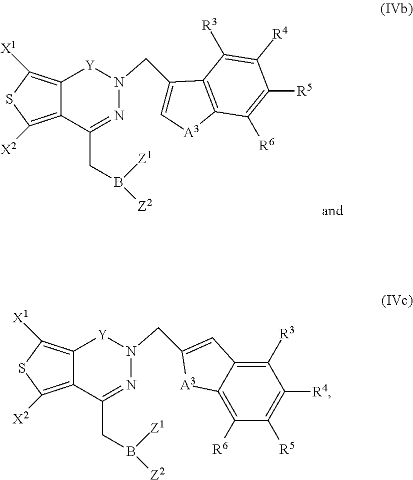

wherein,

Z¹ and Z² are independently selected from the group consisting of hydroxy, alkoxy, aryloxy, or Z¹ and Z² taken together with the boron atom to which they are bonded form

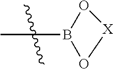

wherein,

X is a substituted or unsubstituted C₂-C₅ alkylene.

In certain embodiments, R³ through R⁶ of Formula (IV) are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl.

In certain embodiments, R³ through R⁶ of Formula (IV) are independently hydrogen, halogen or haloalkyl. In certain embodiments, R³ through R⁶ are independently hydrogen, halogen or trihaloalkyl.

In certain embodiments, R³ and R⁶ of Formula (IV) are hydrogen. In certain embodiments, R³, R⁵, and R⁶ are hydrogen.

In certain embodiments, R⁴ of Formula (IV) is hydrogen, halogen or haloalkyl. In certain embodiments, R⁴ is hydrogen. In certain embodiments, R⁴ is halogen. In certain embodiments, R⁴ is haloalkyl. In certain embodiments, R⁴ is CF₃.

In certain embodiments, R³ through R⁶ of Formula (IV) are hydrogen. In certain embodiments, R³, R⁵, R⁶ are hydrogen and R⁴ is halogen or haloalkyl. In certain embodiments, R³, R⁵, R⁶ are hydrogen and R⁴ is haloalkyl. In certain embodiments, R³, R⁵, R⁶ are hydrogen and R⁴ is CF₃. In certain embodiments, R³, R⁵, R⁶ are hydrogen and R⁴ is halogen. In certain embodiments, R³, R⁵, R⁶ are hydrogen and R⁴ is F. In certain embodiments, R³, R⁵, R⁶ are hydrogen and R⁴ is Cl.

In certain embodiments, Y of Formula (IV) is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A¹ of Formula (IV) is NR⁷, O, S or CH₂. In certain embodiments, A¹ is NR⁷, O, or S. In certain embodiments, A¹ is NR⁷, S or CH₂. In certain embodiments, A¹ is NR⁷ or O. In certain embodiments, A¹ is NR⁷ or S. In certain embodiments, A¹ is NR⁷. In certain embodiments, A¹ is O. In certain embodiments, A¹ is S.

In certain embodiments, A² of Formula (IV) is N or CH. In certain embodiments, A² is N. In certain embodiments, A² is CH.

In certain embodiments, A³ of Formula (IV) is NR⁷, O, or S. In certain embodiments, A³ is O. In certain embodiments, A³ of Formula (IV) is S. In certain embodiments, A³ is NR⁷.

In certain embodiments, X¹ and X² of Formula (IV) are hydrogen.

In certain embodiments, X¹ and X² of Formula (IV) are halogen. In certain embodiments, X¹ and X² are Cl.

In certain embodiments, X¹ and X² of Formula (IV) are independently hydrogen or halogen. In certain embodiments, X¹ is hydrogen and X² is Cl. In certain embodiments, X¹ is Cl and X² is hydrogen.

In certain embodiments, Z of Formula (IV) is

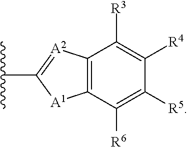

In certain embodiments, Z of Formula (IV) is

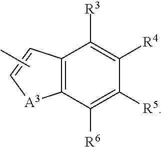

In certain embodiments, R⁷ of Formula (IV) is hydrogen, C₁-C₄ alkyl, or C(O)O-(C₁-C₄)-alkyl. In certain embodiments, R⁷ is hydrogen. In certain embodiments, R⁷ is C₁-C₄ alkyl. In certain embodiments, R⁷ is C₁-C3 alkyl. In certain embodiments, R⁷ is C₁-C2 alkyl. In certain embodiments, R⁷ is C₁-C₄ n-alkyl. In certain embodiments, R⁷ is C₁-C3 n-alkyl. In certain embodiments, R⁷ is C(O)O-(C₁-C₄)-alkyl. In certain embodiments, R⁷ is C(O)O-(C₁-C₃)-alkyl. In certain embodiments, R⁷ is C(O)O-(C₁-C2)-alkyl. In certain embodiments, R⁷ is C(O)O- (C₁-C₄)-n-alkyl. In certain embodiments, R⁷ is C(O)O-(C₁-C₃)-n-alkyl.

In certain embodiments, the compounds of Formula (IV) is

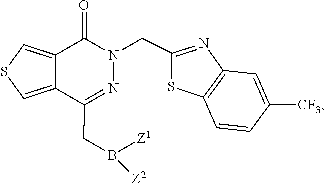

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

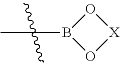

wherein,

X is a substituted or unsubstituted C₂-C₅ alkylene.

In certain embodiments, the compounds of Formula (IV) is

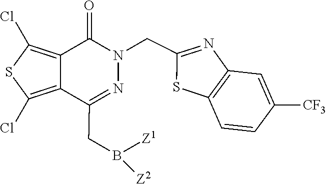

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

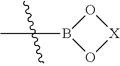

wherein,

X is a substituted or unsubstituted C₂-C₅ alkylene.

In certain embodiments, the compounds of Formula (IV) is

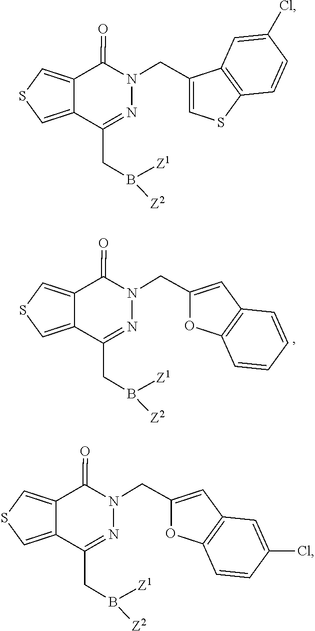

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

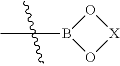

wherein,

X is a substituted or unsubstituted C₂-C₅ alkylene.

In certain embodiments, the compounds of Formula (IV) is

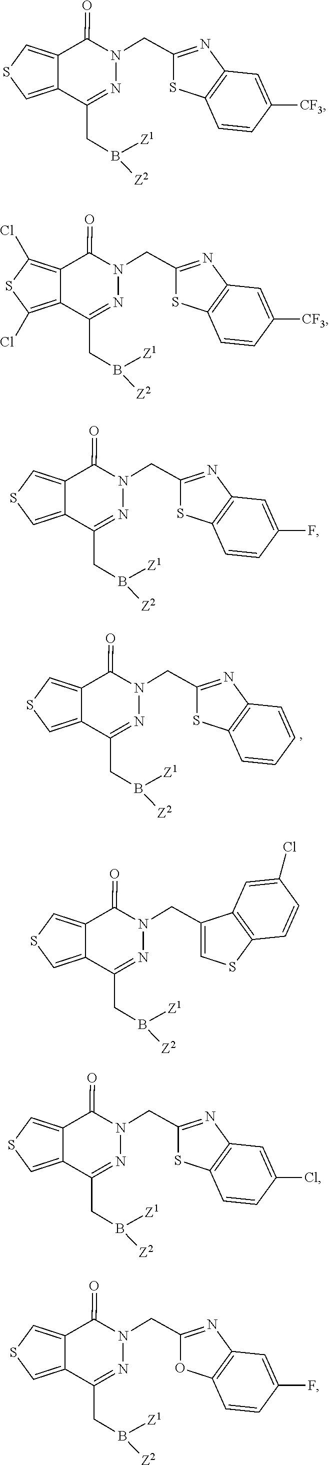

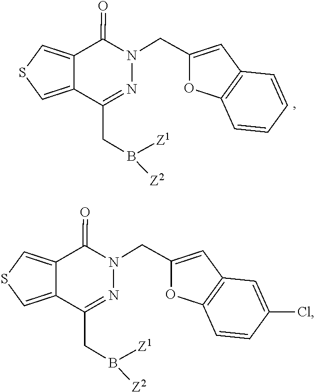

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

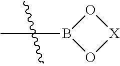

wherein,

X is a substituted or unsubstituted C₂-C₅ alkylene.

In another aspect, the aldose reductase inhibitor is a compound of Formula (V)

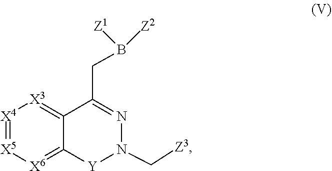

or pharmaceutically acceptable salts, pro-drugs or solvates thereof;

wherein,

X³ is N or CR⁸;

X⁴ is N or CR⁹;

X⁵ is N or CR¹⁰;

X⁶ is N or CR¹¹; with the proviso that two or three of X³, X⁴, X⁵, or X⁶ are N;

Z¹ and Z² are independently selected from the group consisting of hydroxy, alkoxy, aryloxy, or Z¹ and Z² taken together with the boron atom to which they are bonded form

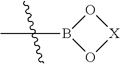

wherein,

X is a substituted or unsubstituted C₂-C₅ alkylene;

Z³ is

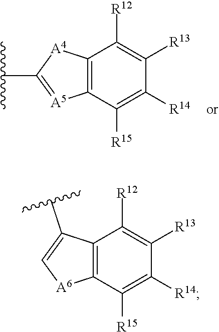

A⁴ is NR¹⁶, O, S or CH₂;

A⁵ is N or CH;

A⁶ is NR¹⁶, O, or S;

R⁸ through R¹⁵ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R⁸ through R¹¹ or two of R¹² through R¹⁵ taken together are (C₁-C₄)-alkylenedioxy; and

R¹⁶ is hydrogen, C₁-C₄ alkyl, or C(O)O-(C₁-C₄)-alkyl.

Suitable substituents on the C₂-C₅ alkylene include one or more alkyl, alkoxy, aryl, aryloxy, halo, haloalkyl, haloalkoxy, haloalkylthio. A preferred substituted C₂-C₅ alkylene is substituted ethylene. A more preferred substituted C₂-C₅ alkylene is —C(CH₃)₂C(CH₃)₂—.

It will be recognized by those of skill in the art that the designation of
Z is

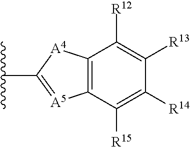

### or Z is

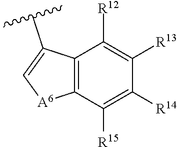

indicates that when Z is

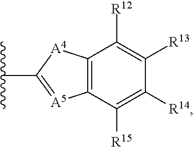

the compounds of Formula (V) are understood to encompass

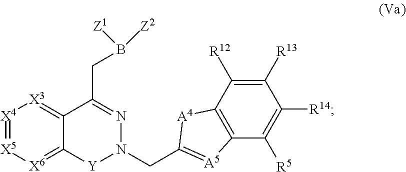

and when Z is

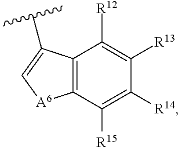

the compounds of Formula (V) are understood to encompass

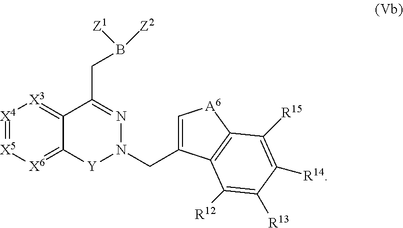

In some compounds of Formula (V), R⁸ through R¹⁵ are independently hydrogen, halogen or haloalkyl, for example, R⁸ through R¹⁵ are independently hydrogen, halogen or trihaloalkyl (e.g., —CF₃).

In other compounds of Formula (V), R⁸ through R¹¹ are hydrogen.

In certain embodiments of compounds of Formula (V), R¹² through R¹⁵ are independently hydrogen, halogen or haloalkyl, for example, R¹² through R¹⁵ are independently hydrogen, halogen or trihaloalkyl (e.g., —CF₃).

In certain embodiments, R¹² and R¹⁵ of Formula (V) are hydrogen.

In certain embodiments, R¹³ of Formula (V) is hydrogen, halogen or haloalkyl. In certain embodiments, R¹³ is hydrogen. In certain embodiments, R¹³ is halogen. In certain embodiments, R¹³ is haloalkyl.

In certain embodiments, R¹⁴ of Formula (V) is hydrogen, halogen or haloalkyl. In certain embodiments, R¹⁴ is hydrogen. In certain embodiments, R¹⁴ is halogen. In certain embodiments, R¹⁴ is haloalkyl.

In certain embodiments, Y of Formula (V) is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A⁴ of Formula (V) is NR¹⁶, S or CH₂. In certain embodiments, A⁴ is NR¹⁶ or O. In certain embodiments, A⁴ is NR¹⁶ or S. In certain embodiments, A⁴ is NR¹⁶. In certain embodiments, A⁴ is O. In certain embodiments, A⁴ is S.

In certain embodiments, A⁵ of Formula (V) is N or CH. In certain embodiments, A⁴ is N. In certain embodiments, A⁴ is CH.

In certain embodiments, A⁶ of Formula (V) is O or S. In certain embodiments, A⁶ is O. In certain embodiments, A⁶ is S.

In certain embodiments, X³ and X⁶ of Formula (V) are nitrogen.

In certain embodiments, X³ and X⁴ of Formula (V) are nitrogen.

In certain embodiments, X³ and X⁵ of Formula (V) are nitrogen.

In certain embodiments, X⁴ and X⁵ of Formula (V) are nitrogen.

In certain embodiments, X⁴ and X⁶ of Formula (V) are nitrogen.

In certain embodiments, X⁵ and X⁶ of Formula (V) are nitrogen.

In certain embodiments, Z³ of Formula (V) is

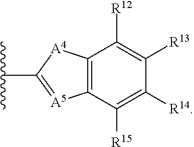

In certain embodiments, Z³ of Formula (V) is

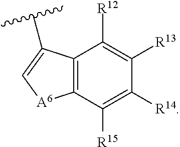

In some embodiments, the compounds of Formula (V) is

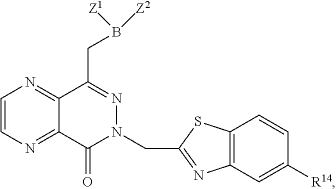

or pharmaceutically acceptable salts, pro-drugs or solvates thereof;

wherein,

R¹⁴ is hydrogen, halogen or trihaloalkyl (e.g., —CF₃); and

Z¹ and Z² are independently selected from the group consisting of hydroxy, alkoxy, aryloxy, or Z¹ and Z² taken together with the boron atom to which they are bonded form

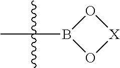

wherein,

X is a substituted or unsubstituted C₂-C₅ alkylene.

In embodiments, the compounds of Formula (V) is

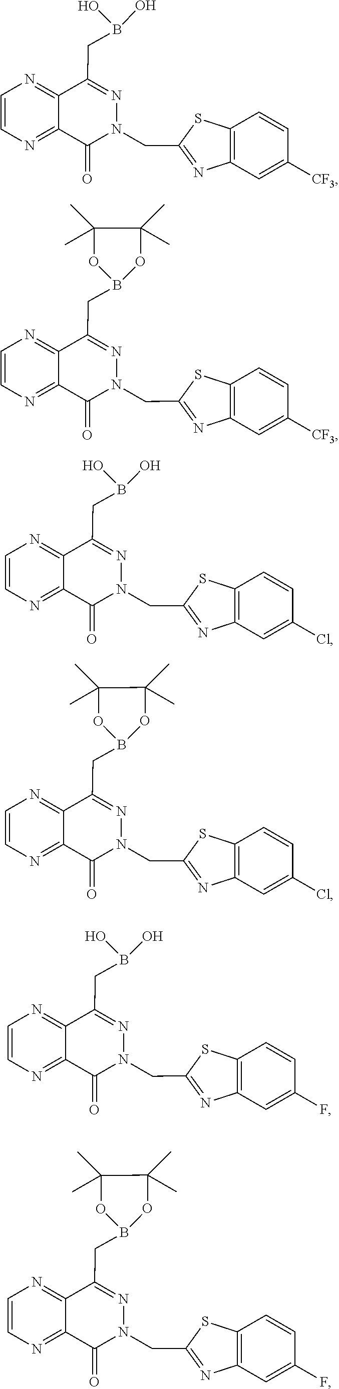

or pharmaceutically acceptable salts, pro-drugs or solvates thereof.

In one aspect, the aldose reductase inhibitor is a compound of Formula (VI)

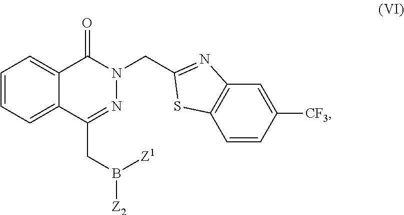

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

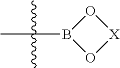

wherein,

X is a substituted or unsubstituted C₂-C₅ alkylene.

In an embodiment, the aldose reductase inhibitor of Formula (VI) is

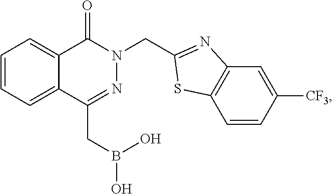 [SMILES: O=c1c2ccccc2c(CB(O)O)nn1Cc1nc2cc(C(F)(F)F)ccc2s1]

or pharmaceutically acceptable salts, pro-drugs or solvates thereof.

In an embodiment, the AH inhibitor of Formula (VI) is

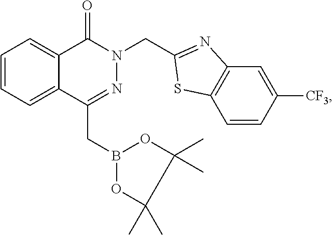 [SMILES: CC1(C)OB(Cc2nn(Cc3nc4cc(C(F)(F)F)ccc4s3)c(=O)c3ccccc23)OC1(C)C]

or pharmaceutically acceptable salts, pro-drugs or solvates thereof.

The term “alkyl”, as used herein, unless otherwise indicated, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, where the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “halogen” or “halo-”, as used herein, means chlorine (Cl), fluorine (F), iodine (I) or bromine (Br).

As used herein, the term “acyl” is used in a broad sense to designate radicals of the type RCO—, in which R represents an organic radical which may be an alkyl, aralkyl, aryl, alicyclic or heterocyclic radical, substituted or unsubstituted, saturated or unsaturated; or, differently defined, the term “acyl” is used to designate broadly the monovalent radicals left when the OH group of the carboxylic radical is removed from the molecule of a carboxylic acid.

The term “alkoxy” is employed to designate a group of the formula: —O—R wherein R is an alkyl group, which optionally contains substituents, such as halogen. Preferably, the term “alkoxy” is employed to designate an alkoxy with an alkyl group of 1 to 6 carbon atoms. Most preferably, the term “alkoxy” is employed to designate an alkoxy with an alkyl group of 1 to 3 carbon atoms, such as methoxy or ethoxy.

The term “cycloalkyl group” is used herein to identify cycloalkyl groups having 3-6 carbon atoms preferably cyclopropyl, cyclobutyl, cyclopentyl and cyclohexyl.

The term “solvate” as used herein means a compound, or a pharmaceutically acceptable salt thereof, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

A “prodrug” refers to an agent, which is converted into the parent drug in vivo. Prodrugs are often useful because, in some situations, they are easier to administer than the parent drug. They are bioavailable, for instance, by oral administration whereas the parent drug is either less bioavailable or not bioavailable. The prodrug also has improved solubility in pharmaceutical compositions over the parent drug. For example, the compound carries protective groups which are split off by hydrolysis in body fluids, e.g., in the bloodstream, thus releasing active compound or is oxidized or reduced in body fluids to release the compound. The term “prodrug” may apply to such functionalities as, for example; the acid functionalities of the compounds of Formula (I). Prodrugs may be comprised of structures wherein an acid group is masked, for example, as an ester or amide. Further examples of prodrugs are discussed herein. See also Alexander et al. (J. Med. Chem. 1988, 31, 318), which is incorporated by reference. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, and biohydrolyzable phosphate analogues. Prodrugs are also described in, for example, The Practice of Medicinal Chemistry (Camille Wermuth, ed., 1999, Academic Press; hereby incorporated by reference in its entirety). In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh; each of which hereby incorporated by reference in its entirety). Biohydrolyzable moieties of a compound of Formula I (a) do not interfere with the biological activity of the compound but can confer upon that compound advantageous properties in vivo, such as uptake, duration of action, or onset of action; or (b) may be biologically inactive but are converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, a-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

The term “salt” includes salts derived from any suitable of organic and inorganic counter ions well known in the art and include, by way of example, hydrochloric acid salt or a hydrobromic acid salt or an alkaline or an acidic salt of the aforementioned amino acids. The term is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids; and salts derived from inorganic or organic bases including, for example sodium, potassium, calcium, ammonium or tetrafluoroborate. Exemplary pharmaceutically acceptable salts are found, for example, in Berge, et al, (J. Pharm. Sci. 1977, 66(1), 1; and U.S. Pat. Nos. 6,570,013 and 4,939,140; each hereby incorporated by reference in its entirety). Pharmaceutically acceptable salts are also intended to encompass hemi-salts, wherein the ratio of compound: acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

The term “acid” contemplates all pharmaceutically acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically acceptable aliphatic, alicyclic and aromatic carboxylic acids, dicarboxylic acids, tricarboxylic acids, and fatty acids. Preferred acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, fumaric acid, acetic acid, propionic acid, isopropionic acid, valeric acid, alpha-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tartaric acid and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

### III. Compositions

The compounds can be administered in the form a suitable composition, such as a pharmaceutical composition. Pharmaceutical compositions are physiologically acceptable and typically include the active compound and a carrier. The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002); each of which hereby incorporated by reference in its entirety).

The composition can be in a desired form, such as a table, capsule, solution, emulsion, suspension, gel, sol, or colloid that is physiologically and/or pharmaceutically acceptable. If desired, the carrier can include a buffer, for example with alkaline buffers, e.g., ammonium buffer, acidic buffers, e.g., ethanoates, citrates, lactates, acetates, etc., or zwitterionic buffers, such as, glycine, alanine, valine, leucine, isoleucine and phenylalanine, Kreb's-Ringer buffer, TRIS, MES, ADA, ACES, PIPES, MOPSO, cholamine chloride, MOPS, BES, TES, HEPES, DIPSO, MOBS, TAPSO, acetamidoglycine, TEA, POPSO, HEPPSO, EPS, HEPPS, Tricine, TRIZMA, Glycinamide, Glycyl-glycine, HEPBS, Bicine, TAPS, AMPB, CHES, AMP, AMPSO, CAPSO, CAPS, and CABS.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. If desired tonicity adjusting agents can be included, such as, for example, sugars, sodium chloride or combinations thereof. In some embodiments, the composition is isotonic.

The compositions may also include additional ingredients, such as acceptable surfactants, co-solvents, emollients, agents to adjust the pH and osmolarity and/or antioxidants to retard oxidation of one or more component.

The compositions can be prepared for administration by any suitable route such as ocular (including periocular and intravitreal administration), oral, parenteral, intranasal, anal, vaginal, topical, subcutaneous, intravenous, intra-arterial, intrathecal and intraperitoneal administration. Accordingly, while intrathecal administration is an option and may be selected by a clinician (e.g., when the aldose reductase inhibitor is not central nervous system penetrant), it is generally preferred that the aldose reductase inhibitor is not administered intrathecally. Oral compositions may be incorporated directly with the food of the diet. Preferred carriers for oral administration comprise inert diluents, edible carriers or combinations thereof. Examples of pharmaceutically acceptable carriers may include, for example, water or saline solution, polymers such as polyethylene glycol, carbohydrates and derivatives thereof, oils, fatty acids, or alcohols. Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N R′R″R′″R″″Y″, in which the R radicals are identical or different optionally hydroxylated hydrocarbon radicals and Y″ is an anion of a strong acid, such as halide, sulfate and sulfonate anions; cetyltrimethylammonium bromide is one of the cationic surfactants which can be used, amine salts of formula NR′R′R″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

If desired, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc., or combinations thereof containing two or more of the foregoing.

Additional formulations which are suitable for other modes of administration include suppositories. Moreover, sterile injectable solutions may be prepared using an appropriate solvent. Generally, dispersions are prepared by incorporating the various sterilized amino acid components into a sterile vehicle, which contains the basic dispersion medium and/or the other ingredients. Suitable formulation methods for any desired mode of administration are well known in the art (see, generally, Remington's Pharmaceutical Sciences, 18^{th} Ed. Mack Printing Company, 1990).

Typical pharmaceutically acceptable compositions can contain a an AR inhibitor and/or a pharmaceutically acceptable salt thereof at a concentration ranging from about 0.01 to about 2 wt%, such as 0.01 to about 1 wt% or about 0.05 to about 0.5 wt%. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like. Other equivalent modes of administration can be found in U.S. Pat. No. 4,939,140.

When administered to a subject, the AR inhibitor and pharmaceutically acceptable carriers can be sterile. Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain minor amounts of wetting or emulsifying agents, or pH buffering agents.

The pharmaceutical formulations of the present disclosure are prepared by methods well-known in pharmaceutics. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

In some embodiments, the composition is in unit dose form such as a tablet, capsule or single-dose vial. Suitable unit doses, i.e., therapeutically effective amounts, may be determined during clinical trials designed appropriately for each of the conditions for which administration of a chosen compound is indicated and will, of course, vary depending on the desired clinical endpoint.

Any of the compounds and/or compositions of the disclosure may be provided in a kit comprising the compounds and/or compositions. Thus, in one embodiment, the compound and/or composition of the disclosure is provided in a kit comprising in the same package or separate package, a carrier and optionally instructions for using the kit for therapeutic or prophylactic end usage.

### IV. Combination Therapy

The methods described herein include the administration of an AR inhibitor and one more additional therapeutic agents. The additional therapeutic agents may be administered before, concurrently with or after the AR inhibitor, but in a manner that provides for overlap of the pharmacological activity of the AR inhibitor and the additional therapeutic agent. The additional therapeutic agent can be, for example, second aldose reductase inhibitor, an antioxidant, or both.

For example, the 2^{nd} aldose reductase can be a compound described in, for example, in U.S. Pat. Nos. 5,677,342; 5,155,259; 4,939,140; U.S. 2006/0293265; and Roy et al., (Diabetes Research and Clinical Practice, 10, Issue 1, 91-97, 1990; and references cited therein; each of which hereby incorporated by reference in its entirety. Aldose reductase inhibitors include, for example, zopolrestat, epalrestat, ranirestat, berberine and sorbinil, as described in, e.g., U.S. Pat. Nos. 4,939,140; 6,159,976; and 6,570,013. Preferably, the 2^{nd} aldose reductase inhibitor is selected from ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210.

Other therapeutic agents that can be administered include, for example corticosteroids, e.g., prednisone, methylprednisolone, dexamethasone, or triamcinalone acetinide, or noncorticosteroid anti-inflammatory compounds, such as ibuprofen or flubiproben,. Similarly, vitamins and minerals, e.g., zinc, and micronutrients can be co-administered. In addition, inhibitors of the protein tyrosine kinase pathway, which include natural protein tyrosine kinase inhibitors like quercetin, lavendustin A, erbstatin and herbimycin A, and synthetic protein tyrosine kinase inhibitors like tyrphostins (e.g., AG490, AG17, AG213 (RG50864), AG18, AG82, AG494, AG825, AG879, AG1112, AG1296, AG1478, AG126, RG13022, RG14620 and AG555), dihydroxy-and dimethoxybenzylidene malononitrile, analogs of lavendustin A (e.g., AG814 and AG957), quinazolines (e.g., AG1478), 4,5-dianilinophthalimides, and thiazolidinediones, can be co-administered with genistein or an analog, prodrug or pharmaceutically acceptable salt thereof (see Levitzki et al., Science 267: 1782-1788 (1995); and Cunningham et al., Anti-Cancer Drug Design 7: 365-384 (1992)). In this regard, potentially useful derivatives of genistein include those set forth in Mazurek et al., U.S. Pat. No. 5,637,703. Selenoindoles (2-thioindoles) and related disulfide selenides, such as those described in Dobrusin et al., U.S. Pat. No. 5,464,961, are useful protein tyrosine kinase inhibitors. Neutralizing proteins to growth factors, such as a monoclonal antibody that is specific for a given growth factor, e.g., VEGF (for an example, see Aiello et al., PNAS USA 92: 10457-10461 (1995)), or phosphotyrosine (Dhar et al., Mol. Pharmacol. 37: 519-525 (1990)), can be co-administered. Other various compounds that can be co-administered include inhibitors of protein kinase C (see, e.g., U.S. Pat. Nos. 5,719,175 and 5,710,145), cytokine modulators, an endothelial cell-specific inhibitor of proliferation, e.g., thrombospondins, an endothelial cell-specific inhibitory growth factor, e.g., TNFα, an anti-proliferative peptide, e.g., SPARC and prolferin-like peptides, a glutamate receptor antagonist, aminoguanidine, an angiotensin-converting enzyme inhibitor, e.g., angiotensin II, calcium channel blockers, y-tectorigenin, ST638, somatostatin analogues, e.g., SMS 201-995, monosialoganglioside GM1, ticlopidine, neurotrophic growth factors, methyl-2,5-dihydroxycinnamate, an angiogenesis inhibitor, e.g., recombinant EPO, a sulphonylurea oral hypoglycemic agent, e.g., gliclazide (non-insulin-dependent diabetes), ST638 (Asahi et al., FEBS Letter 309: 10-14 (1992)), thalidomide, nicardipine hydrochloride, aspirin, piceatannol, staurosporine, adriamycin, epiderstatin, (+)-aeroplysinin-1, phenazocine, halomethyl ketones, anti-lipidemic agents, e.g., etofibrate, chlorpromazine, spinghosines and retinoic acid and analogs thereof (Burke et al., Drugs of the Future 17 (2): 119-131 (1992); and Tomlinson et al., Pharmac. Ther. 54: 151-194 (1992)).

The present disclosure further provides for the use of the compounds of Formula (I)-(VI), or a pharmaceutically acceptable salt, hydrate, solvate, or prodrug thereof, in a method of treating a disease state, and/or condition caused by or related to PMM2-CDG. In another embodiment, the disclosure relates to use of the compounds of Formula (I)-(VI), or a pharmaceutically acceptable salt, hydrate, solvate, or prodrug thereof, in a method of treating a disease state, and/or condition caused by or related to PMM2-CDG, comprising the steps of: (a) identifying a subject in need of such treatment; (b) providing a compound of Formula (I)-(VI), or a pharmaceutically acceptable salt, hydrate, solvate, prodrug thereof; and (c) administering said compound of Formula (I)-(VI) in a therapeutically effective amount to treat, suppress and/or prevent the disease state or condition in a subject in need of such treatment.

In another embodiment, the disclosure relates to use of the compounds of Formula (I)-(VI), or a pharmaceutically acceptable salt, hydrate, solvate, or prodrug thereof, in a method of treating a disease state, and/or condition caused by or related to PMM2-CDG, comprising the steps of: (a) identifying a subject in need of such treatment; (ii) providing a composition comprising a compound of Formula (I)-(VI), or a pharmaceutically acceptable salt, hydrate, solvate, prodrug or tautomer thereof; and (iii) administering said composition in a therapeutically effective amount to treat, suppress and/or prevent the disease state or condition in a subject in need of such treatment.

In the aforementioned embodiments, the compound or composition is preferably used orally.

### V. Example—In vitro Activation of PMM2 by Compound B

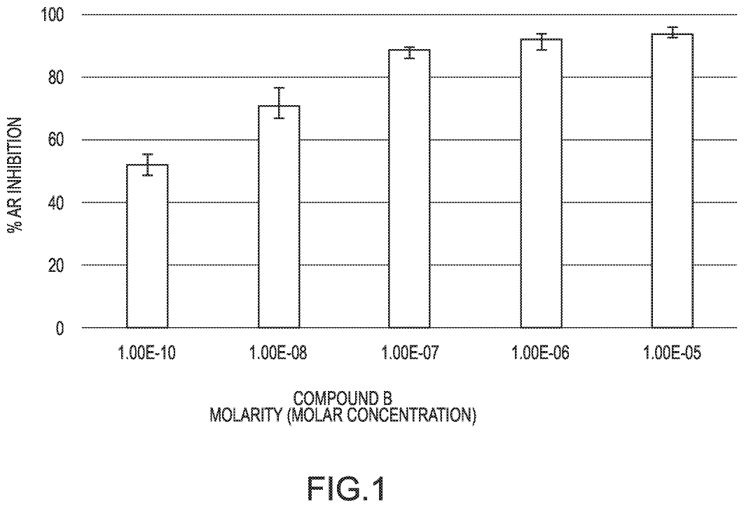

The in vitro activation of PMM2 by Compound B was studied in PMM2-CDG patient-derived fibroblasts. Compound B is a potent and selective inhibitor of aldose reductase. The AR inhibiting activity of Compound B was demonstrated in a microplate assay using D-glyceraldehyde and NADPH as substrate for aldose reductase in the presence of Compound B at concentrations ranging from 0.1 nM to 10 μM. The results, presented as a percent inhibition of maximal activity, are summarized in FIG. 1. It is noted that compared to epalrestat, which has a reported mean AR inhibition concentration [IC₅₀] of 72 nM, Compound B (IC₅₀=0.10 nM) is a significantly more potent inhibitor of Aldose Reductase enzymatic activity.

Since there are negative effects on the central nervous system in PMM2-CDG patients, Compound B which is a CNS penetrant Aldose Reductase inhibitor was tested in fibroblast cell lines derived from four unique, individual PMM2-CDG patients in order to determine if PMM2 enzyme activation could be detected.

Cells were seeded in a 96-well plate, homogenization buffer (20 mM HEPES, 25 mM KCl, 1 mM DTT, 10 μg/ml leupeptin, 10 μg/ml antipain) was added and plates were freeze-thawed at —80° C. twice to lyse cells. Reaction buffer (50 mM HEPES, 5 mM MgCl₂, 0.5 mM NADP+, 10 μg/ml yeast glucose-6-phosphate dehydrogenase, 10 μM glucose-1,6-bisphosphate, 10 μg/ml phosphoglucoisomerase, 5.25 μg/ml phosphomannoseisomerase) containing 200 μM mannose-1-phosphate as substrate was then added to the wells of each plate. Plates were incubated at 37° C. for 270 min and absorbance was read at 340 nm at 30, 60, 90, 120, 150, 180, 210, 240 and 270 min by removing the plate from incubation at the respective time points. All incubations were carried out with or without substrate (mannose-1-phosphate) and the difference between the two values was calculated as the enzymatic activity. Enzyme activity was normalized to total lysate protein levels. Enzyme activities of fibroblasts in the absence of an AR inhibitor were determined. To assess the effect of Compound B on enzyme activity, Compound B was incubated with the cell line at a concentration of 50 nM for a period of 24 hours. Following this, enzyme activity was assessed as described above. At least two biological replicates were conducted and enzyme activity in the presence of Compound B was compared with that of a DMSO-treated mutant cell line used as the control. For ease of analysis, the enzyme activity (as represented by NADPH concentration) of each treatment condition was compared with the activity of a baseline, untreated mutant cell line at the last time point.

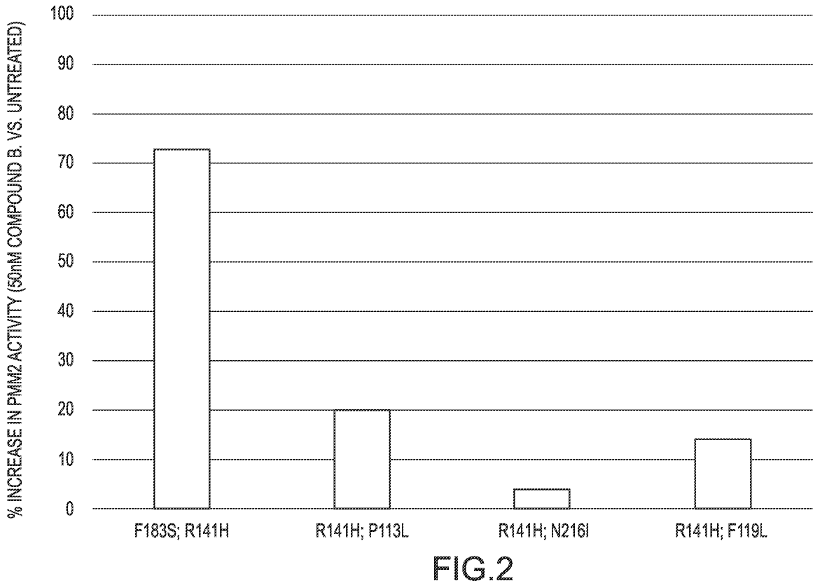

The results of these studies are shown in FIG. 2. Each individual patient's heterozygous amino acid substitutions are shown beneath each bar of the graph in FIG. 2. Compound B increased PMM2 enzyme activity in each of the four patient-derived cell lines tested. Compound B was shown to be a potent activator of PMM2 activity in PMM2-CDG patient-derived fibroblasts.

Unless otherwise defined, all technical and scientific terms used herein have the same meaning as commonly understood by one of ordinary skill in the art to which this disclosure belongs.

Although methods and materials similar or equivalent to those described herein can be used in the practice or testing of the present disclosure, suitable methods and materials are described in the foregoing paragraphs. In addition, the materials and methods are illustrative only and not intended to be limiting. All United States patents and published or unpublished United States patent applications cited herein are incorporated by reference. All published foreign patents and patent applications cited herein are hereby incorporated by reference. All published references, documents, manuscripts, scientific literature cited herein are hereby incorporated by reference. All identifier and accession numbers pertaining to scientific databases referenced herein (e.g., PUBMED, NCBI, GENBANK, EBI) are hereby incorporated by reference.

1. A method of treating PMM2-CDG, comprising administering a therapeutically effective amount of an aldose reductase inhibitor to a subject in need thereof.

2. A method of increasing PMM2 enzymatic activity in a subject with PMM2-CDG, comprising administering a therapeutically effective amount of an aldose reductase inhibitor to the subject.

3. The method of claim 1, wherein the aldose reductase inhibitor is a compound of Formula (III): 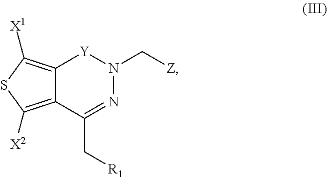
or a salt thereof, wherein,
R¹ is CO₂R²;
R² is H, (C₁-C₆)-alkyl, (C₁-C₆)-hydroxyalkyl, or (C₁-C₆)-aminoalkyl;
X¹ is H or halogen;
X² is H or halogen;
Y is a bond, C═O, C═S, C═NH, or C═N(C₁-C₄)-alkyl;
Z is 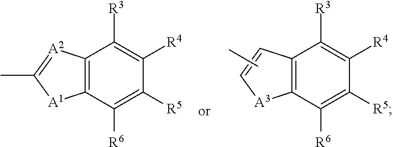
A¹ is NR⁷, O, S or CH₂;
A² is N or CH;
A³ is NR⁷, O, or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and
R⁷ is hydrogen, C₁-C₄ alkyl, or C(O)O-(C₁-C₄)-alkyl.

4. The method of claim 3, wherein the aldose reductase inhibitor is selected from the group consisting of: 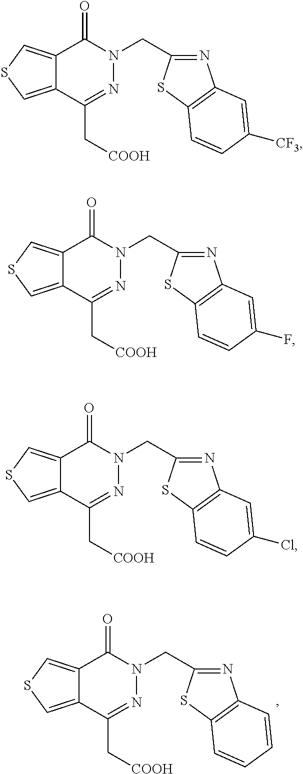 [SMILES: O=C(O)Cc1nn(Cc2nc3cc(C(F)(F)F)ccc3s2)c(=O)c2cscc12 ; O=C(O)Cc1nn(Cc2nc3cc(F)ccc3s2)c(=O)c2cscc12 ; O=C(O)Cc1nn(Cc2nc3cc(Cl)ccc3s2)c(=O)c2cscc12 ; O=C(O)Cc1nn(Cc2nc3ccccc3s2)c(=O)c2cscc12]
and salts thereof.

5. The method of claim 1, wherein the aldose reductase inhibitor is 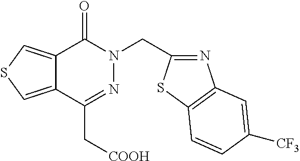 [SMILES: O=C(O)Cc1nn(Cc2nc3cc(C(F)(F)F)ccc3s2)c(=O)c2cscc12]
or a salt thereof.

6. The method of claim 1, wherein the aldose reductase inhibitor is a compound of Formula (II):
or a salt thereof, wherein: 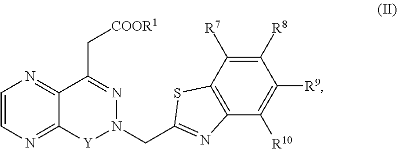
R¹ is H, (C₁-C₆)-alkyl, (C₁-C₆)-hydroxyalkyl, or (C₁-C₆)-aminoalkyl;
Y is a bond, C═O, C═S, C═NH, or C═N(C₁-C₄)-alkyl; and
R⁷ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R⁷ through R¹⁰ taken together are (C₁-C₄)-alkylenedioxy.

7. The method of claim 6, wherein the aldose reductase inhibitor is selected from the group consisting of: 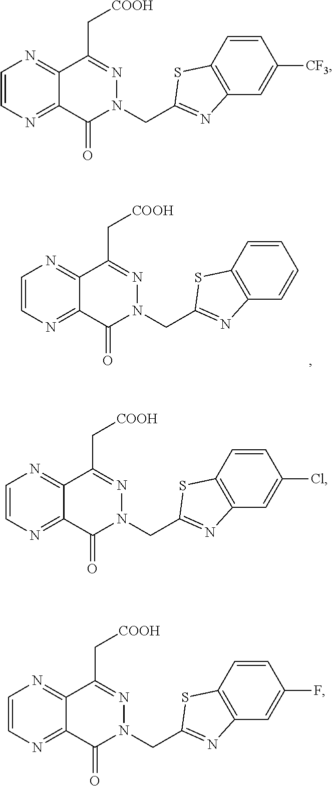 [SMILES: O=C(O)Cc1nn(Cc2nc3cc(C(F)(F)F)ccc3s2)c(=O)c2nccnc12 ; O=C(O)Cc1nn(Cc2nc3ccccc3s2)c(=O)c2nccnc12 ; O=C(O)Cc1nn(Cc2nc3cc(Cl)ccc3s2)c(=O)c2nccnc12 ; O=C(O)Cc1nn(Cc2nc3cc(F)ccc3s2)c(=O)c2nccnc12]
and salts thereof.

8. The method of claim 7, wherein the aldose reductase inhibitor is 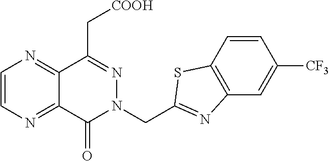 [SMILES: O=C(O)Cc1nn(Cc2nc3cc(C(F)(F)F)ccc3s2)c(=O)c2nccnc12]
or a salt thereof.

9. (canceled)

10. The method of claim 1, wherein the subject is a human.

11-19. (canceled)

20. The method of claim 2, wherein the aldose reductase inhibitor is a compound of Formula (III): 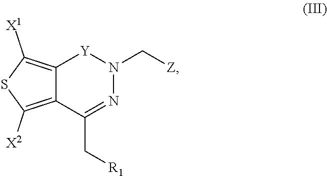
or a salt thereof, wherein,
R¹ is CO₂R²;
R² is H, (C₁-C₆)-alkyl, (C₁-C₆)-hydroxyalkyl, or (C₁-C₆)-aminoalkyl;
X¹ is H or halogen;
X² is H or halogen;
Y is a bond, C═O, C═S, C═NH, or C═N(C₁-C₄)-alkyl;
Z is 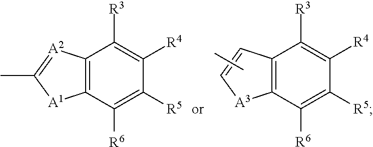
A¹ is NR⁷, O, S or CH₂;
A² is N or CH;
A³ is NR⁷, O, or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkyl sulfonyl; and
R⁷ is hydrogen, C₁-C₄ alkyl, or C(O)O-(C₁-C₄)-alkyl.

21. The method of claim 20, wherein the aldose reductase inhibitor is selected from the group consisting of: 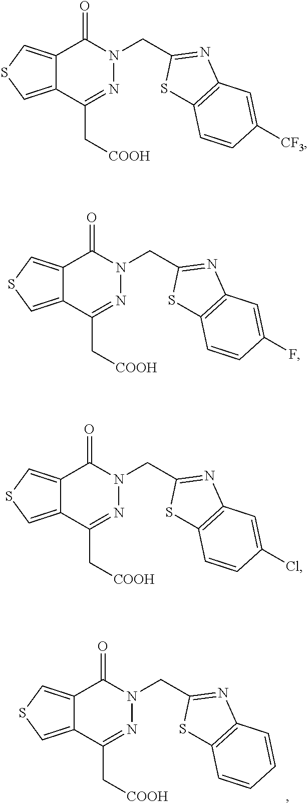 [SMILES: O=C(O)Cc1nn(Cc2nc3cc(C(F)(F)F)ccc3s2)c(=O)c2cscc12 ; O=C(O)Cc1nn(Cc2nc3cc(F)ccc3s2)c(=O)c2cscc12 ; O=C(O)Cc1nn(Cc2nc3cc(Cl)ccc3s2)c(=O)c2cscc12 ; O=C(O)Cc1nn(Cc2nc3ccccc3s2)c(=O)c2cscc12]
and salts thereof.

22. The method of claim 2, wherein the aldose reductase inhibitor is 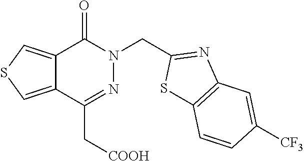 [SMILES: O=C(O)Cc1nn(Cc2nc3cc(C(F)(F)F)ccc3s2)c(=O)c2cscc12]
or a salt thereof.

23. The method of claim 2, wherein the aldose reductase inhibitor is a compound of Formula (II): 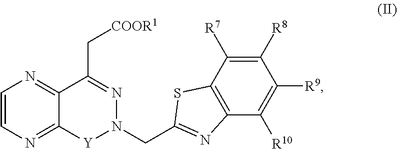
or a salt thereof, wherein:
R¹ is H, (C₁-C₆)-alkyl, (C₁-C₆)-hydroxyalkyl, or (C₁-C₆)-aminoalkyl;
Y is a bond, C═O, C═S, C═NH, or C═N(C₁-C₄)-alkyl; and
R⁷ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R⁷ through R¹⁰ taken together are (C₁-C₄)-alkylenedioxy.

24. The method of claim 23, wherein the aldose reductase inhibitor is selected from the group consisting of: 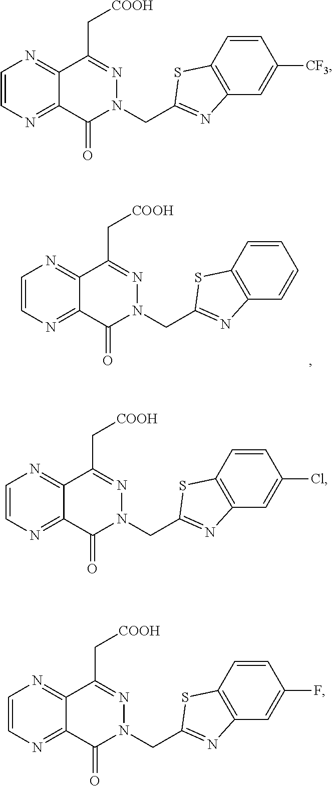 [SMILES: O=C(O)Cc1nn(Cc2nc3cc(C(F)(F)F)ccc3s2)c(=O)c2nccnc12 ; O=C(O)Cc1nn(Cc2nc3ccccc3s2)c(=O)c2nccnc12 ; O=C(O)Cc1nn(Cc2nc3cc(Cl)ccc3s2)c(=O)c2nccnc12 ; O=C(O)Cc1nn(Cc2nc3cc(F)ccc3s2)c(=O)c2nccnc12]
and salts thereof.

25. The method of claim 24, wherein the aldose reductase inhibitor is 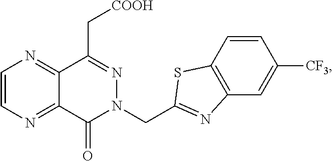 [SMILES: O=C(O)Cc1nn(Cc2nc3cc(C(F)(F)F)ccc3s2)c(=O)c2nccnc12]
or a salt thereof.

26. The method of claim 1, wherein the aldose reductase inhibitor is a compound of Formula (I): 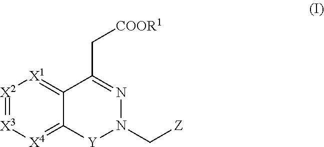
or a salt thereof, wherein:
R¹ is H, (C₁-C₆)-alkyl, (C₁-C₆)-hydroxyalkyl, or (C₁-C₆)-aminoalkyl;
X¹ is N or CR³;
X² is N or CR⁴;
X³ is N or CR⁵;
X⁴ is N or CR⁶; with the proviso that two or three of X¹, X², X³, or X⁴ are N;
Y is a bond, C═O, C═S, C═NH, or C═N(C₁-C₄)-alkyl;
Z is 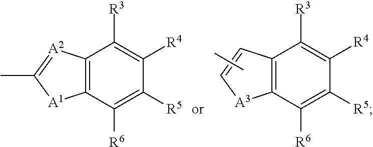
A¹ is NR¹¹, O, S or CH₂;
A² is N or CH;
A³ is NR¹¹, O, or S;
R³ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R³ through R⁶ or two of R⁷ through R¹⁰ taken together are (C₁-C₄)-alkylenedioxy; and
R¹¹ is hydrogen, C₁-C₄ alkyl, or C(O)O-(C₁-C₄)-alkyl.

27. The method of claim 2, wherein the aldose reductase inhibitor is a compound of Formula (I): 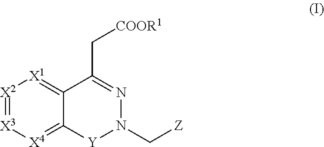
or a salt thereof, wherein:
R¹ is H, (C₁-C₆)-alkyl, (C₁-C₆)-hydroxyalkyl, or (C₁-C₆)-aminoalkyl;
X¹ is N or CR³;
X² is N or CR⁴;
X³ is N or CR⁵;
X⁴ is N or CR⁶; with the proviso that two or three of X¹, X², X³, or X⁴ are N;
Y is a bond, C═O, C═S, C═NH, or C═N(C₁-C₄)-alkyl;
Z is 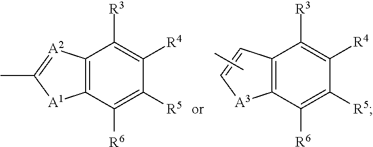
A¹ is NR¹¹, o, S or CH₂;
A² is N or CH;
A³ is NR¹¹, O, or S;
R³ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R³ through R⁶ or two of R⁷ through R¹⁰ taken together are (C₁-C₄)-alkylenedioxy; and
R¹¹ is hydrogen, C₁-C₄ alkyl, or C(O)O-(C₁-C₄)-alkyl.

28. The method of claim 2, wherein the subject is a human.
